(19) United States
(12) Patent Application Publication
(10) Pub. No.: US 2008/0089993 A1
(43) Pub. Date: Apr. 17, 2008

(54) SYSTEM AND METHOD FOR MODIFYING A FLUID FOR ORAL ADMINISTRATION

(75) Inventors: Charles G. Hwang, Ridgewood, NJ (US); Rebecca C. Eaton, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Remote Clinical Solutions, Inc., San Francisco, CA (US)

(21) Appl. No.: 11/676,228

(22) Filed: Feb. 16, 2007

Related U.S. Application Data
(63) Continuation-in-part of application No. PCT/US2005/030146, filed on Aug. 23, 2005.
(60) Provisional application No. 60/603,949, filed on Aug. 23, 2004.

(51) Int. Cl. A23L 2/52; A61B 5/00

(52) U.S. Cl. 426/590; 600/307

(57) ABSTRACT

Methods and systems are provided for modifying a fluid for oral administration. Typical systems include a reservoir which contains or may be filled with water or other aqueous components. The systems also include a container or retention pocket which is integral with or mountable on or in the reservoir. The container holds a soluble beneficial agent, typically an electrolyte, a nutrient or a medicament. The container is adapted to selectively combine a measured amount of the beneficial agent with the aqueous component based upon patient need.

### CROSS-REFERENCES TO RELATED APPLICATIONS

This application is a continuation-in-part of PCT/US2005/030146 (Attorney Docket No. 022337-000210PC), filed on Aug. 23, 2005, which claimed the benefit and priority of U.S. Provisional Patent Application No. 60/603,949 (Attorney Docket 022337-000200US), filed Aug. 23, 2004, the full disclosures of which are hereby incorporated by reference for all purposes.

### BACKGROUND OF THE INVENTION

This application relates to a method and system for modifying a fluid for oral administration and, further, to a method and system for providing a hydration fluid to a patient.

Certain populations are particularly at risk for developing various fluid and electrolyte disorders—among them, hypernatremia (elevated blood sodium levels), hyponatremia (depleted blood sodium levels), volume depletion, and volume overload-including independent seniors (for whom dehydration ranks among the top five most frequent causes for hospitalization), institutionalized seniors (of whom over 50 percent acquire hypo- or hypernatremia in any given 12-month period), young children (for whom dehydration resulting from gastroenteritis accounts for 10 percent of pediatric hospital admissions), post-surgical hospital patients (of whom between 5 percent and 15 percent develop hyper- or hyponatremia), professional and non-professional athletes (for whom dehydration of as little as 2 percent (dehydration of between 5 and 10 percent is common) can reduce athletic performance by as much as 20 percent), chronically-ill individuals (a number of chronic conditions, or medications for such conditions, including diabetes and hypertension, precipitate dehydration), military personnel (the Military Operational Medical Research Program has characterized fluid and electrolyte imbalances and dehydration as among the highest non-adversarial threats to U.S. superiority in the battlespace), and mining and forestry personnel. Dehydration can lead to a number of serious medical complications, including renal failure, heart failure, brain damage, heat stroke, and death. If not treated in a timely fashion, mortality rates may exceed 50 percent. In 2000, the costs associated with dehydration-related hospitalizations among the 65+ demographic alone totaled $3.8 billion.

Dehydration, or risk thereof, is extraordinarily difficult to monitor. First, severe dehydration can occur very rapidly, in just a couple of hours. Second, many of the symptoms associated with dehydration (e.g. fatigue, confusion, dry mouth) do not appear until substantial fluids have been lost and medical complications take hold. Finally, many of the symptoms of dehydration may be present among normally-hydrated, at-risk individuals (among seniors, for example, a number of chronic conditions, and medications for such conditions, cause confusion; among athletes, anaerobic exercise often causes dry mouth and/or fatigue). The implication of the latter is that individuals at risk for dehydration, or their health care providers, often attribute classic signs of dehydration to non-hydration-related conditions and do not seek to correct the condition as a result.

Even more importantly, perhaps, fluid and electrolyte disorders are extraordinarily difficult to correct, as fluid loss and the ratio of fluid-to-electrolyte loss—both critical to understanding the amount, and concentration, of the oral or intravenous hydration solution required to correct these disorders—are unknown. Individuals are left to formulate their own “best-guess” estimates of fluid and electrolyte replacement needs. These best-guess estimates are rarely accurate, as the type and degree of fluid and electrolyte loss vary dramatically across patient populations and intra-patient over time depending upon a number of different variables, including heat, humidity, altitude, sweat rate, management of various chronic conditions, diet, fluid consumption, weight, sex, race, and age, among other variables. Comprehensive diets and frequent fluid administration often fall short of preventing the onset of various fluid and electrolyte disorders in at-risk populations.

The field of hydration monitoring and rehydration therapy is active. Its importance lies in facilitating early detection and correction. Ideally, hydration monitoring would be near continuous and non-invasive. Information would be available to direct and enable a closed loop system in which electrolytes would be automatically delivered to an aqueous solution for oral administration so as to keep serum fluid and electrolyte levels close to normal physiological levels. Such a system would reduce medical complications and provide obvious increases in quality of life for at-risk patients.

It is known that information derived from biometric data, for example analyte levels in body fluids, may be employed to reliably predict the onset of, or to indicate the presence of, a fluid or electrolyte disorder in a human patient. For example, for patients presenting symptoms of fluid or electrolyte disorders, physicians will often order lab tests which measure any of a number of different clinical parameters in body fluids—most often in blood or urine—including, among others: sodium concentrations, osmolality, blood urea nitrogen (BUN) levels, creatinine levels, BUN/creatinine ratios, hematocrit levels, protein levels, glucose levels, keytone levels, amylase levels, calcium levels, urate levels, chloride levels, albumin levels, and urine specific gravity. Other non-analyte measures used to improve the accuracy of diagnosis and to guide rehydration therapy include weight change, mucous membrane moistness, reported renal function, urine volume, urine color, tissue turgor, venous pressure, postural change in heart rate, postural change in blood pressure, body temperature, respiratory rate, heat rate, blood pressure, medication and medical history, recent environmental conditions (e.g. heat, exercise, etc.), recent change in functional ability (e.g. cognitive function, continence, etc.), fever/diarrhea/vomiting, and recent fluid intake. Most systems designed to determine the concentration of one or multiple analytes in body fluids employ electronic, chemical or electrochemical methods such as disclosed in U.S. Pat. Nos. 6,752,927; 6,743,597; 6,689,618; 6,635,491; 6,602,719; 6,503,198; 6,403,384, 6,149,865; 6,087,088; 6,068,971; 5,837,546; and 5,766,870.

A major drawback of such systems is that they require medically trained personnel to interpret the results and to translate the results of the clinical tests into fluid replacement recommendations. Even if a medical professional is available to provide such recommendations based on clinical lab data, patients operating in an outpatient setting cannot easily translate clinical guidelines into fluid compositions that adhere to such guidelines. That is, commercially-available oral rehydration solutions fix the concentrations of beneficial agents at the time of manufacture. The latter means that while some consumers of such commercially-available rehydration solutions are ingesting too high a concentration of electrolytes given their serum analyte concentrations, other consumers are ingesting too low a concentration of electrolytes given their serum analyte concentrations.

Prior art discloses various drug delivery systems, and beverage containers, which recognize the need for separating beneficial agents from liquid components until just prior to oral administration including, among others, U.S. Pat. Nos. 6,685,692; 6,652,134; 6,541,055; 6,382,411; 6,354,190; 6,269,973; 5,921,955; and 5,125,534. However, such systems combine substantially all of the beneficial agents with the liquid component, thereby providing no means for controlling the amount of the beneficial agent to be delivered, or the concentration of the resulting mixture.

Prior art discloses liquid dosing devices for beverage or liquid chemical manufacture, whereby the amount of beneficial agents to be delivered to the liquid component is variable and based upon measured parameters of the combined beneficial agent and component mixture, including U.S. Pat. Nos. 6,387,424; 6,129,104; 5,816,448; 5,234,134; 5,154,319; and 5,058,780. In contrast, the disclosed invention varies the amount of beneficial agents to be delivered based upon measured patient biometric data.

Prior art discloses drug delivery systems, which vary the beneficial agent to be delivered based upon patient biometric data including, among others, U.S. Pat. Nos. 6,572,542; 6,558,351; 6,309,373; 6,024,090, which describe insulin delivery systems, among others, that control insulin dosage based upon measured blood glucose levels. The aforementioned patents, among others, describe methods of delivery including inhalation, injection/infusion and subcutaneous administration, whereas the present invention describes delivery via oral administration.

It will now be seen that there exists a need for a system that receives patient biometric data indicative of a patient's condition, for a method for rapidly processing this data to determine a patient's need with respect to a beneficial agent, and for a system that delivers a controlled amount of the agent in response to such need. Furthermore, there exists a need for a system that receives data indicative of a patient's current fluid and/or electrolyte levels, for a method for rapidly processing this data to determine fluid and/or electrolyte replacement and, potentially, maintenance needs, and for a system that delivers a controlled amount of at least one beneficial agent in response to such replacement and maintenance needs. At least some of these objectives will be fulfilled by the present invention.

### BRIEF SUMMARY OF THE INVENTION

The present invention provides both methods and systems for providing soluble beneficial agents to patients, particularly including providing electrolytes or other hydration agents in water and other aqueous solutions for drinking. Apparatus according to the present invention comprise drinking systems which include a reservoir which contains or may be filled with water or other aqueous components. The systems also include a container or retention pocket which is integral with, mounted on, or may be mountable on or in, the reservoir. The container holds a soluble beneficial agent, typically an electrolyte or other hydration agent or a medicament. The container is adapted to selectively combine a measured amount of the beneficial agent with the aqueous component.

The reservoir of the drinking system may have any form suitable for holding the aqueous component and subsequently mixing the aqueous component with the beneficial agent. Usually, the reservoir will permit sealing and storage of the aqueous component for extended periods of time. Exemplary reservoirs include cups, bottles, bladders, tubes, boxes, cans, pouches, covered children's cups, on-demand drinking apparatuses, and the like. The container will usually be formed as part of the reservoir or be selectively attachable to, or detached from, the reservoir. For example, the container may comprise a plurality of dosage or dose-releasing receptacles which are formed as part of the reservoir. The patient or other user may then selectively release the doses of beneficial agent into the aqueous component from the receptacles in the reservoir. More commonly, however, the container will be removably attached to the reservoir, typically being attached to a spout or other component or feature on the reservoir from which the patient or other user may drink. For example, the spout may form a typical threaded connector of the type found on plastic water bottles. The container may then comprise a holder having a complementary threaded end for attachment to the reservoir. Typically, the container will also include a mechanism for selectively delivering a measured or calibrated amount of the beneficial agent within the container. After releasing the beneficial agent from the container, the container may be removed and the patient or other user may then drink from the bottle through the spout.

A number of different mechanisms may be provided for selectively combining the contents of the container with the aqueous component within the reservoir. For example, a mechanical element, such as a pusher plate or a pusher bar, can be mounted on the container to selectively advance and dispense a calibrated amount of the beneficial agent from the container. Alternatively, a mechanical element, such as a blister mechanism which can be depressed against a pierceable material to release measured amounts of the beneficial agent from the container, can be employed. In such instances, the beneficial agent may be in the form of a powder or granulated solid, a plurality of encapsulated pellets or dissolvable tablets or other discrete dosage forms, a liquid, a gel, or any other form of the agent which may be selectively released in measured amounts. Alternatively, the beneficial agent may be held in a pouch, osmotic device, or other holder from which it may be squeezed or otherwise pushed. The selective amount may then be released based on the amount of the pouch which is squeezed or the osmotic gradient. Other systems may employ dials, levers, measuring receptacles, or any other variety of mechanisms which can release dosages of the beneficial agent based on user input.

The present invention further provides methods for hydrating patients. The methods rely on determining a level of hydration in the patient and preparing a hydration fluid by combining an amount of the soluble hydration agent with an aqueous component. It may be appreciated that level of hydration may include level of dehydration and level of overhydration. Particularly, the methods provide that the amount of the hydration agent to be combined is selected based on the determined level of hydration. The patient's hydration may be measured by any conventional technique, typically being based upon measured sodium concentration or osmolality, or markers therefor, in saliva, oral fluids, sweat, tears, breath, urine, blood, transudates or exudates. Additional patient data—including the rate or amount of fluid loss, rate or amount of electrolyte loss, fluid electrolyte concentration, fluid osmolality, prior or expected future fluid consumption, prior or expected future electrolyte consumption, weight, weight gain or loss, body mass index, sex, age, race, height behavioral data, diet, fitness level, physical exertion level, physical appearance, cognitive capability, environmental data such as temperature, humidity, altitude, the level of other disease markers, medication and medical procedure history, the presence, absence or severity of one or more particular medical conditions, and, any combination of these—may be used, to refine the calculation of hydration level. Patient averages—including total body water as a percentage of total body weight, rate or amount of fluid loss, rate or amount of electrolyte loss, and fluid electrolyte concentration or osmolality—patient preferences, and previously-recorded recorded patient biometric data and/or trends in previously-recorded patient biometric data may also be used to further refine the calculation of hydration level. The hydration fluid is then prepared by mechanically releasing the calibrated amount of the hydration agent into the aqueous component, typically in a drinking vessel as described above. That is, usually, the hydration agent will be in a container integral with or attached to the drinking vessel. After dispensing the selected amount of the hydration agent into the drinking vessel, the container may or may not be removed.

The present invention fulfills many objects. That is, the present invention provides solutions to problems existing in the prior art. The present invention provides a system for titrating beneficial-agent delivery based on actual beneficial-agent needs, thus combining oral delivery therapies for administering beneficial agents with monitoring technologies so as to effect tight control over the analyte level of a patient. More specifically, the present invention provides a system for titrating fluid and electrolyte delivery based on actual fluid and electrolyte replacement and, potentially, maintenance needs, thus combining oral delivery therapies for administering fluid and electrolytes with monitoring technologies so as to effect tight control over the fluid and electrolyte level of a patient. The optimal hydration solution concentration varies widely from patient to patient, and intra-patient over time, and may be based on a number of different factors. The system of the present invention can deliver all or any proportional amount of the beneficial agents contained in the at least one container, enabling the oral delivery of controlled amounts of beneficial agents based on the particular needs of the patient as determined by measured patient biometric data, or as determined by the latter in combination with patient averages, additional patient data, previously-recorded patient data or trends in previously-recorded patient data, or patient preferences. The present invention also controls the fluid and/or electrolyte state of the patient by determining dosage at the point of consumption, rather than at the point of manufacture, thereby decreasing the incidence of electrolyte disorders resulting from the oral consumption of a solution too high or too low in electrolytes relative to the patient's replacement and maintenance needs.

Various embodiments of the present invention have advantages, including one or more of the following: (a) improving the direct or indirect control that may be exercised over the fluid and electrolyte levels of a patient; (b) quickly delivering the required number and amount of beneficial agents to a patient before a fluid and electrolyte disorder develops or becomes dangerous; (c) overcoming the deficiencies of relying on “best guess” estimates of fluid and electrolyte replacement requirements, either or both of which are often under- or overestimated by patients and formal and informal health care providers; (d) reducing the number and severity of medical complications, thereby increasing patient safety and lowering health care costs due to better control of patient fluid and electrolyte levels.

Various embodiments of the present invention have certain features. In one embodiment of the present invention, the patient or healthcare provider measures saliva sodium concentration, for example, and the information derived from such concentration is used to determine the patient's fluid and electrolyte replacement needs. To further inform the calculation of fluid and electrolyte replacement and/or maintenance needs, the patient or health care provider may input patient averages, including total body water as a percentage of total body weight, fluid electrolyte concentration or osmolality, rate or amount of fluid loss, and rate or amount of electrolyte loss, additional patient data including the rate or amount of fluid loss, rate or amount of electrolyte loss, fluid electrolyte concentration, fluid osmolality, prior or expected future fluid consumption, prior or expected future electrolyte consumption, weight, weight gain or loss, body mass index, sex, age, race, height, behavioral data, diet, fitness level, physical exertion level, physical appearance cognitive capability, environmental data such as temperature, humidity, and altitude, the level of other disease markers, medication and medical procedure history, the presence, absence or severity of one or more particular medical conditions, and any combination of these; or patient preferences including the type or amount of beneficial agent to be delivered to the reservoir. In this embodiment, the control strategy of the system is mechanically based, whereby the patient or health care provider manually manipulates the system based on the patient's current fluid and electrolyte levels, such manipulation causing the delivery of controlled amounts of beneficial agents to the container's reservoir based on the patient's replacement and maintenance needs.

Alternatively, the present invention may comprise a closed loop system in which a diagnostic or monitoring device either external, or integral, to the system of the present invention wirelessly or electronically transmits measured patient biometric data to the first receiving system, which in turn generates a set of commands for the delivery system. In the case of the latter, for example, a saliva-based diagnostic device may be built into the mouthpiece of the container for drinking. A patient activates the system by placing his lips on the mouthpiece of the container, such action generates a saliva sodium concentration reading, such reading is transmitted to the first receiving system which, based on the data transmitted from the diagnostic device, in combination with other data entered manually by the patient, sends a series of commands to the delivery system, which then releases a proportional amount of the beneficial agents contained in the container into the reservoir.

In this embodiment, the control strategy of the system is preferably microprocessor based and/or implemented using dedicated electronics. Such a control strategy would enable the delivery system to generate patient data, such as trends in fluid and/or electrolyte needs and/or consumption, which data may be used to further refine future calculations of fluid and/or electrolyte replacement or maintenance needs.

Based on the information disclosed herein in combination with what is known about fluid and electrolyte administration, computer software can be readily developed which can be used in connection with the receiving and delivery systems of the present invention. More specifically, a microprocessor can be programmed so as to deliver precise doses of electrolytes which correspond to the particular needs of the patient based on manually input or wirelessly or electronically transmitted patient biometric data supplied to the microprocessor. Furthermore, the dosing information contained within the microprocessor can be fed to a separate computer and/or diagnostic or monitoring device in order to calculate the best treatment and dosing schedule for the particular patient.

Optimal control of patients' fluid and electrolyte levels implies reducing the long-term threats of renal and cardiovascular complications. The system and method of the present invention constitute a reliable fluid and electrolyte control system that permits enhanced, tight control of patient fluid and electrolyte levels.

Additional objects, advantages, and embodiments of the invention will be realized by the method and system described in the written description and claims hereof, as well as from the appended drawings. It is to be understood that both the foregoing general description and the following detailed description are exemplary and are intended to provide further explanation of the invention claimed.

### BRIEF DESCRIPTION OF THE DRAWINGS

FIG. 1 illustrates an embodiment of a system of the present invention having a reservoir and a container including a spout structure and cap.

FIGS. 2A-2D illustrate embodiments of a portion the container of FIG. 1.

FIGS. 3A-3B illustrate several embodiments of a reservoir.

FIG. 4 illustrates a cross-sectional view of an embodiment of a container having a one-way valve, wherein the container is attachable to a reservoir.

FIG. 5 provides a cross-sectional view of an embodiment of an encapsulated beneficial agent embodiment.

FIG. 6 provides a cross-sectional view of an embodiment of a pierceable vapor barrier.

FIGS. 7A-7B illustrate embodiments of a blister package.

FIG. 8 provides a cross-sectional view of the blister package embodiments of FIGS. 7A-7B.

FIG. 9 illustrates a cross-sectional view of a pierceable vapor barrier embodiment.

FIGS. 10A-10H illustrate various embodiments of the system of the present invention wherein the container has a variety of positions.

FIG. 11 illustrates a sliding/pusher bar dispensing apparatus.

FIG. 12 illustrates a rotating dial dosing embodiment.

FIG. 13 is a flow chart illustrating an embodiment of a method of the present invention.

FIG. 14 illustrates an embodiment of a rotating dial calibrated as a function of a biometric data type.

FIG. 15 provides a top view of the rotating dial of FIG. 14.

FIGS. 16A-16D illustrate an alternative embodiment of the drinking system which utilizes blister packs for selectively releasing a beneficial agent into the aqueous reservoir.

FIGS. 17A-17D illustrate a further alternative embodiment similar to FIGS. 16A-17D except that the blister packs are incorporated into a bottle neck.

### DETAILED DESCRIPTION OF THE INVENTION

FIG. 1 illustrates an embodiment of a system 35 comprising a reservoir 3 and a container 2 mounted on the reservoir 3. In this embodiment, the container 2 comprises a rotational cap 1 which sits atop a structure 50, wherein the structure 50 attaches to the reservoir 3 by a threaded interface. The container 2 has a spout structure adapted for drinking therethrough.

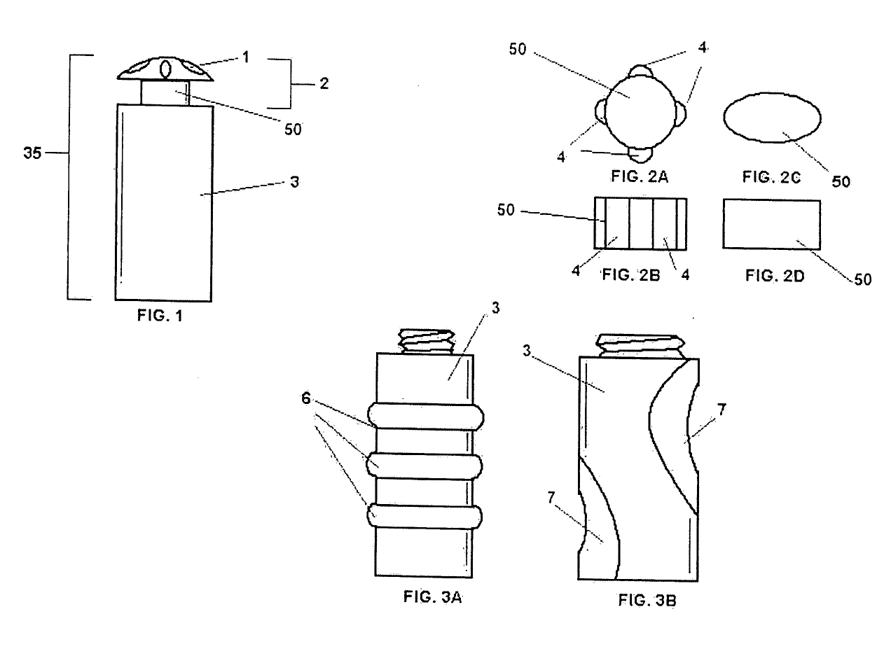

FIGS. 2A-2D provide various views of embodiments of the structure 50 of FIG. 1. FIG. 2A provides a top view of a structure 50 having protrusions 4 radially outwardly, and FIG. 2B illustrates a side view of the structure 50 of FIG. 2A. Embodiments of the structure 50 may have a variety of cross-sectional shapes, including round, oval, square, polygonal, to name a few. FIG. 2C illustrates an embodiment of a structure 50 having a non-circular cross section and FIG. 2D illustrates a side view of the structure 50 of FIG. 2C.

FIG. 3 illustrates various embodiments of reservoirs 3 having gripping features 6 or gripping contoured shapes 7 which give the user a more secure hold on the reservoir 3. This may be particularly useful when agitating the rotational cap 1 or when mixing the dispensed beneficial agents with the aqueous component stored in the reservoir 3.

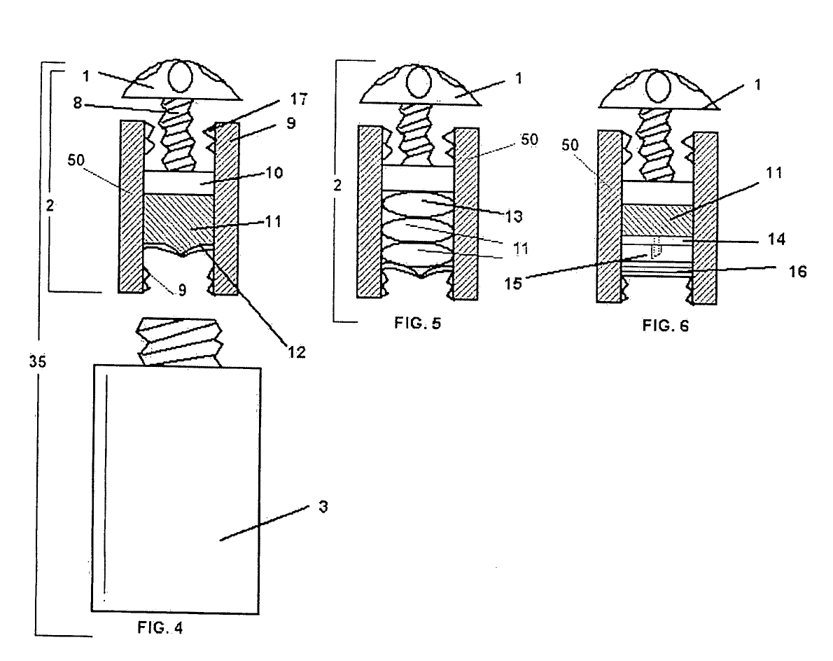

FIG. 4 illustrates an embodiment of a system 35 of the present invention including a container 2 and a reservoir 3. The container 2 includes a rotational cap 1 attached to a threaded protrusion 8, which in turn interfaces with inner diameter threads 17 within structure 50. A barrier component 10 is attached to an end of the thread protrusion 8 distal to the rotational cap 1. When the rotational cap 1 is turned, the barrier component 10 moves axially away from the rotational cap 1, thereby forcing an amount of the beneficial agent 11—proportional to the amount the rotational cap is turned—through a one-way valve 12. When the structure 50 is mounted on the reservoir 3, such as with the use of inner threads 9, the beneficial agent 11 passes into the reservoir 3. When the reservoir 3 holds an aqueous component, the measured amount of agent is selectively combined with the aqueous component.

A variety of beneficial agents 11 may be used. Examples of beneficial agents 11 include electrolytes, rehydration solutions, carbohydrates, nutrients, ergogenic supplements, medicaments, probiotics, diagnostic agents, and clinically and physiologically compatible combinations thereof. When the beneficial agent 11 comprises an electrolyte, the electrolyte may be selected from the group consisting of: particularly sodium and salts of chloride, potassium, calcium, magnesium, bicarbonate, phosphate and sulfate. When the beneficial agent 11 comprises a medicament, the medicament may be selected from the group consisting of: medications for diabetic conditions; chemotherapy agents; gastrointestinal drugs such as antacids; antibiotics; probiotic medications; prokinetic medications; bioactive peptides; antihistamine drugs; anti-infective agents, such as antibiotics, antivirals and urinary tract anti-infectives; antineoplastic agents; autonomic drugs such as adrenergic agents and skeletal muscle relaxants; blood formation and coagulation drugs; cardiovascular drugs; central nervous system agents; diagnostic agents; electrolytic, caloric and water balance agents; enzymes; antitussive, expectorant and mucolytic agents; gold compounds; hormones and synthetic substitutes; smooth muscle relaxants; H.sub.2 blockers like Tagamet.RTM.; consistency-altering agents; unclassified therapeutic agents; and any other medication intended for oral administration.

Another embodiment of a container 2 is illustrated in FIG. 5. Here, the beneficial agent 11 is encapsulated into individual portions, such as capsules 13. The capsule 13 may be coated in a material that would prevent clumping of the beneficial agent 11 due to vapors from the aqueous component stored in the reservoir 3. Encapsulation also provides a means of dividing the beneficial agent 11 into discrete doses.

FIG. 6 illustrates a method for separating the beneficial agent 11 from vapors generated by the aqueous component stored in the reservoir 3. A foil or otherwise pierceable barrier 16 confines the vapor to the reservoir 3. When the rotational cap 1 is turned, the beneficial agent 11 forces a cannulated barrier component 14 toward the pierceable barrier 16. When a cannula 15 extending from the cannulated barrier component 14 pierces the pierceable barrier 16, a channel is created through which the beneficial agent 11, illustrated here in a liquid or gel form, can flow.

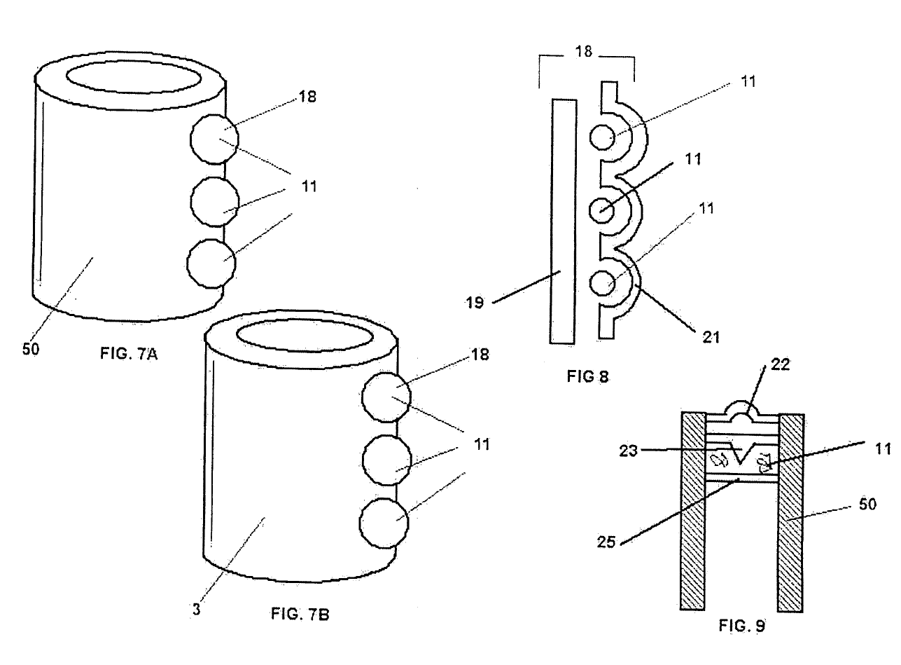

FIGS. 7A, 7B, and 8 illustrate methods and devices for storage and administration of the beneficial agent 11 with the use of a blister mechanism 18. The beneficial agent 11 is stored in a cavity formed by a pierceable material 19 on one side and a deformable material 21 on the other. When the deformable material 21 is depressed, the beneficial agent 11 is forced against the pierceable material 19, which then bursts open, thereby opening a path between the beneficial agent 11 and the aqueous component stored in the reservoir 3. The blister mechanism may be located on or in the container, as in FIG. 7A, or on or in the reservoir, as in FIG. 7B.

FIG. 9 illustrates another embodiment of a pierceable barrier. An elastic component 22 is positioned over a moveable piercing mechanism 23. The beneficial agent 11 is located between the moveable piercing mechanism 23 and a pierceable barrier 25. When the elastic component 22 is depressed, it moves the moveable piercing mechanism 23 forward, piercing the pierceable barrier 25, and enabling the beneficial agent 11 to mix with the aqueous components in the reservoir 3.

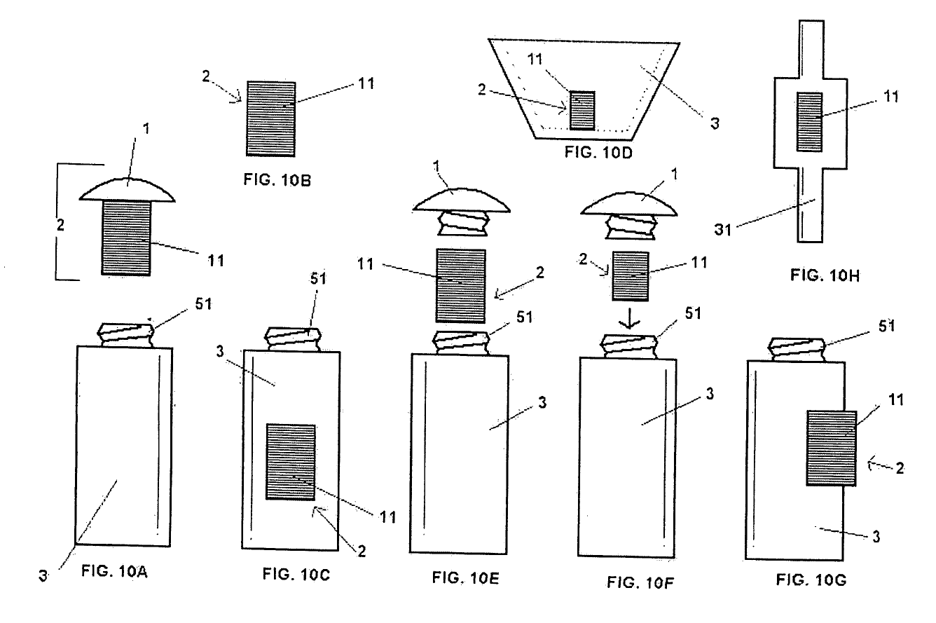

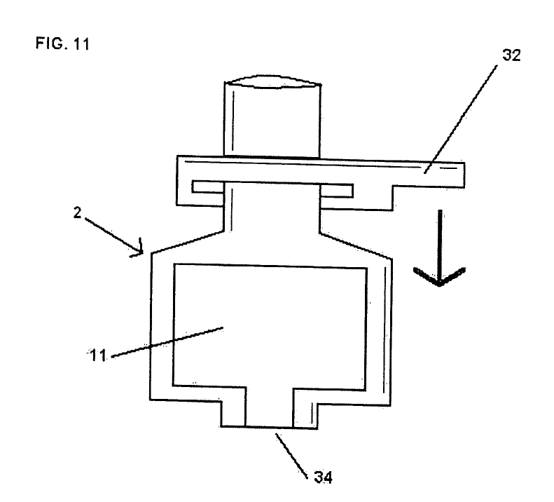

FIGS. 10A-10H illustrate several embodiments of systems of the present invention having the container 2 disposed in various positions. The container 2 holds a soluble beneficial agent 11, and may optionally include other elements including a cap 1, as illustrated in FIG. 10A. Here, the container 2 is separate from the reservoir 3 and attachable to the reservoir 3 with the use of threads 51 on the reservoir 3. FIG. 10B illustrates the container 2 by itself holding a soluble beneficial agent 11. FIG. 10C illustrates the container 2 inside of a reservoir 3. FIG. 10D illustrates a container 2 integral (or attached) to a stand-alone open reservoir 3. FIG. 10E illustrates a container 2 positionable between a cap 1 and a reservoir 3, wherein the container 2 forms a spout structure. FIG. 10F illustrates a container 2 that is advanceable into a reservoir 3, as indicated by an arrow. FIG. 10G illustrates an embodiment wherein the container 2 is integral to the reservoir 3. And, FIG. 10H illustrates an embodiment wherein the container 2 is mounted on or integral with a component or fluid conduit 31.

FIG. 11 illustrates a sliding control mechanism for the dispensing of the beneficial agent 11 from a container 2. Here, the sliding control mechanism comprises a sliding bar 32 having a portion that protrudes such that a user can move the sliding bar 32 relative to the container 2. The movement of the sliding bar 32 pushes the beneficial agent 11 from an exit orifice 34, which allows the beneficial agent 11 to then mix with the aqueous component.

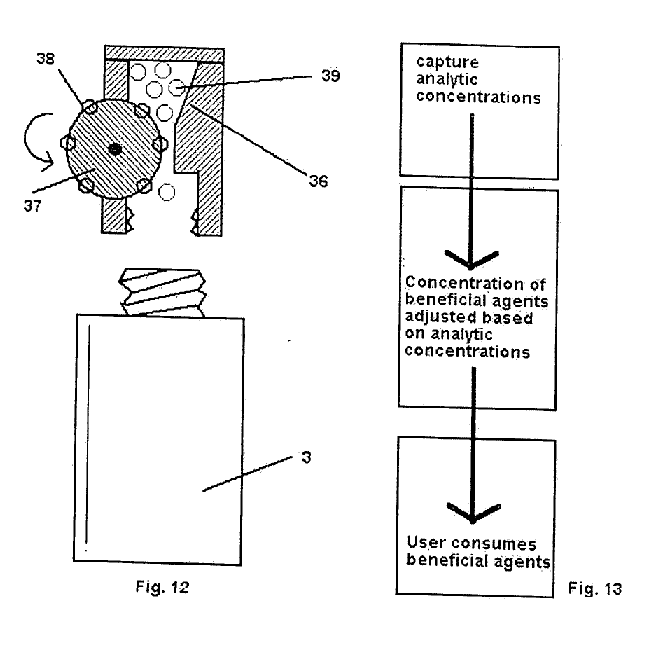

FIG. 12 illustrates a rotating dial dosing mechanism. The rotating dial 37 has external markings, which enable the user to determine how much rotation has been applied to the rotating dial 37. The rotating dial 37 has a series of external protrusions 38, which occlude the beneficial agent channel formed by the rotating dial 37 and the feeder ramp 36. The feeder ramp 36 forces the discrete units 39 of the beneficial agent into a single column. When the rotating dial 37 is turned, a path is temporarily opened for at least one discrete unit 39 to then drop into the reservoir 3.

FIG. 13 uses a flowchart to illustrate an embodiment of a method of the present invention. Here, a patient's biometric data is received and this data is used as an input to automatically adjust the beneficial agent concentration of the aqueous solution to be consumed by the patient.

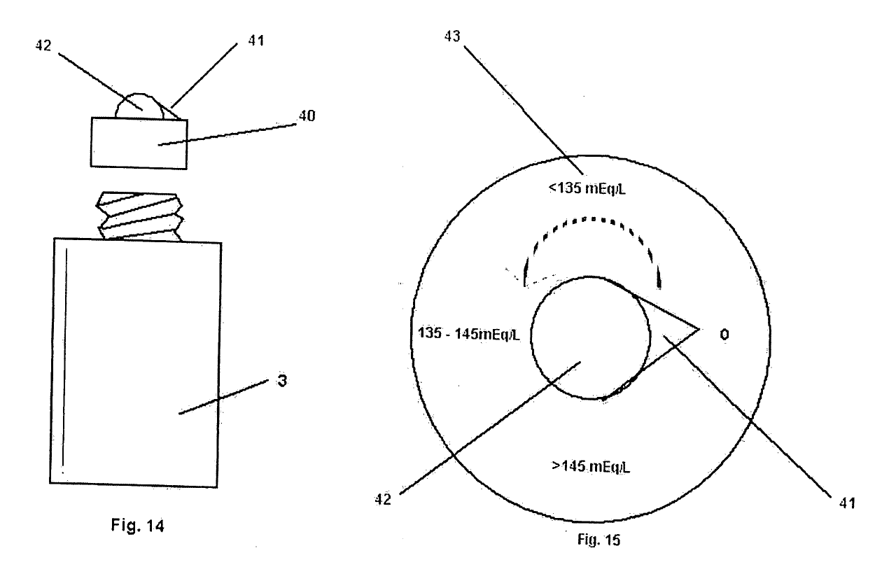

FIG. 14 describes a cap 40 with a twist dial 42 that is used to control the amount of beneficial agent released to the reservoir 3. The twist dial 42 has a protruding pointer 41 that will give the user feedback as to how much beneficial agent is being released.

FIG. 15 describes the relationship of the twist dial 42, the pointer 41, and the calibrations 43. In this embodiment, such calibrations take a numerical form (e.g. the analyte concentration in a bodily fluid in milli-equivalents per liter). As the twist dial 42 is turned, an increasingly or decreasingly proportional amount of the beneficial agent contained in the container is released, and the pointer 41 points to the appropriate corresponding calibration 43.

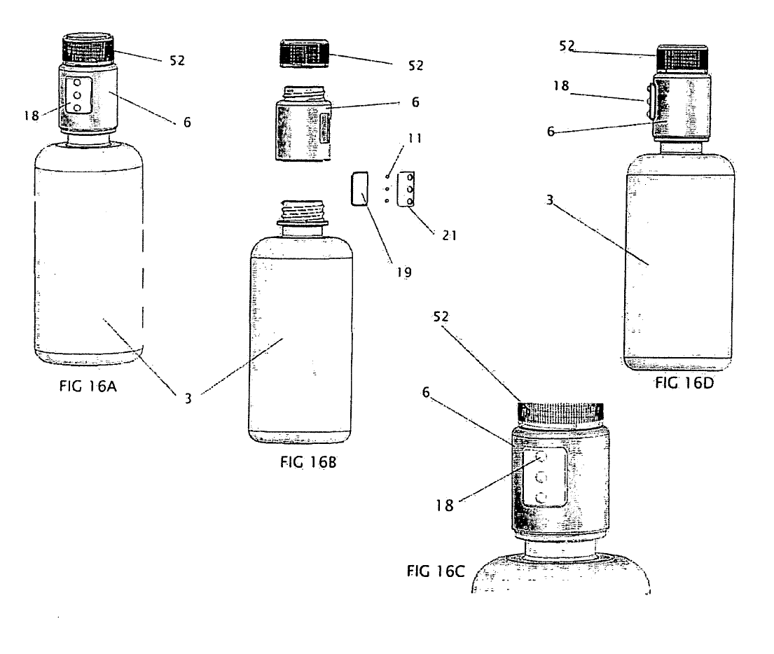

FIGS. 16A, 16B, 16C, and 16D provide additional illustrations of an embodiment of the storage and administration of the beneficial agent 11 with the use of a blister mechanism 18. The beneficial agent 11 is stored in a cavity formed by a pierceable material 19 on one side and a deformable material 21 on the other. The blister mechanism 18, comprised of a pierceable material 19, beneficial agent 11, and deformable material 21, is housed in an enclosure 6 that interfaces with a cap 52 on one end and a reservoir 3 on the other. When the deformable material 21 is depressed, the beneficial agent 11 is forced against the pierceable material 19, which then bursts open, thereby opening a path between the beneficial agent 11 and the aqueous component stored in the reservoir 3.

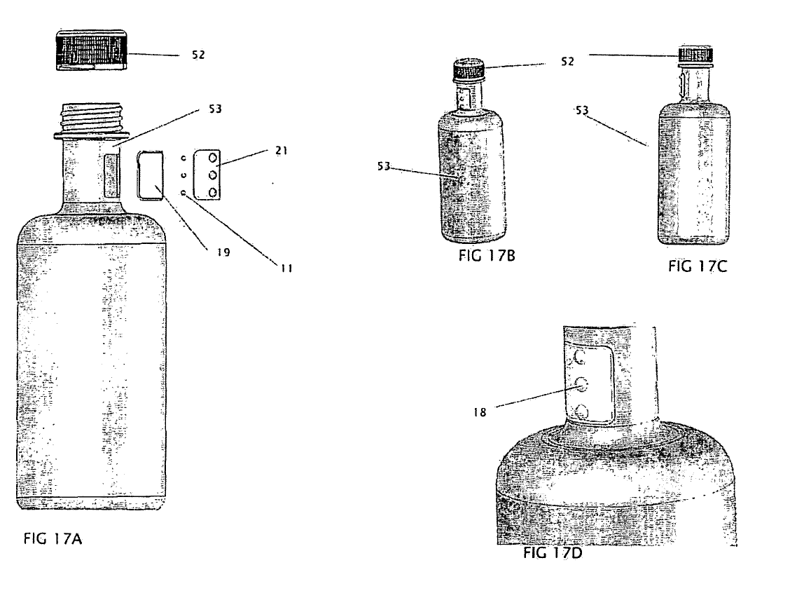

FIGS. 17A, 17B, 17C, and 17D provide additional illustrations of an embodiment of the storage and administration of the beneficial agent 11 with the use of a blister mechanism 18. The beneficial agent 11 is stored in a cavity formed by a pierceable material 19 on one side and a deformable material 21 on the other. The blister mechanism 18, comprised of a pierceable material 19, beneficial agent 11, and deformable material 21, form a subassembly that is attached to an opening in the reservoir 53. When the deformable material 21 is depressed, the beneficial agent 11 is forced against the pierceable material 19, which then bursts open, thereby opening a path between the beneficial agent 11 and the aqueous component stored in the reservoir 53.

An example system for modifying a patient's water and/or electrolyte levels as a function of measured patient biometric data includes a reservoir containing at least one aqueous component, at least one beneficial agent being in a form adapted to be taken up in the aqueous component for oral administration, and at least one container capable of storing the at least one beneficial agent and of separating the at least one beneficial agent from the aqueous component or from vapors of the aqueous component contained in the reservoir. In addition, the system includes a first receiving system configured for receiving a patient's measured biometric data, said receiving system calibrated as a function of at least one biometric data type; a processor which calculates a quantity of the at least one beneficial agent that is to be released to the reservoir utilizing the received measured biometric data; and a delivery system configured to release the quantity of the at least one beneficial agent into the reservoir to form a mixture for oral administration.

The reservoir may have a variety of forms including a bottle, a cup, a bottle, a tube, a box, a can, a sack, a rehydration container, a thermos, a canister, a soft gu-like container, a pouch, a drinking-straw-type tube, a nursing bottle, a covered children's cup, a sippy cup, an on-demand drinking apparatus, or a backpack, to name a few. Typically, the reservoir is comprised of a hard material, a soft material, plastic, glass, aluminum, stainless steel, rubber, or a combination thereof. Further, the reservoir may include a cover, cap and/or a straw. The at least one aqueous component may have a variety of forms including a liquid or semi-liquid medium.

The container may be removably attached to, formed integrally with, appended to, or detached from the reservoir. In some of these embodiments, the reservoir includes a spout-like or cap-like structure and the container is integral with or appended to the spout-like or cap-like structure. In some embodiments, the container resides in between, and threads with, the spout-like or cap-like structure and the reservoir. In some embodiments, the container is formed integrally with the reservoir. In some embodiments, the container is configured to move unrestrained within the reservoir. The container may store the at least one beneficial agent in one or multiple doses. In some embodiments, the container comprises a barrier that separates the beneficial agents from the reservoir, such as a mechanical barrier, an electrical or magnetic field barrier, or a material barrier that changes states. In a mechanical embodiment, the mechanical barrier may comprise a mechanical door, a capsule, a one-way valve, or a pierceable material that is impenetrable to liquid, moisture, or vapors when unpierced. Typically, the mechanical barrier is comprised of plastic, rubber, or aluminum.

The at least one beneficial agent may take a variety of forms including a granulated solid, powder, coated or uncoated granules, an encapsulated tablet, a compressed tablet, a dissolvable tablet, a capsule, a gel, or a liquid. It may be appreciated that the at least one beneficial agent may alternatively or in addition be contained in an osmotic device or in controlled release dosage form units. In some embodiments, the at least one beneficial agent is selected from the group consisting of an electrolyte, a rehydration solution, a carbohydrate, a nutrient, an ergogenic supplement, a medicament, a probiotic, a diagnostic agent, and combinations of these. In some embodiments, the system further includes an electrolyte, carbohydrate, nutrient, ergogenic supplement, medicament, probiotic, or flavoring or coloring agent disposed in a container.

In some embodiments, the biometric data includes at least one of: a measured sodium concentration in a body fluid, a measured osmolality in a body fluid, a protein concentration in a body fluid, a presence of at least one analyte in a body fluid, an absence of at least one analyte in a body fluid, a quantity of at least one analyte in a body fluid, a concentration of at least one analyte in a body fluid, a rate or amount of fluid loss, a rate or amount of electrolyte loss, fluid electrolyte concentration, weight, a body temperature, a saliva flow rate, a sweat rate, a urine volume, a capillary refill time, a mucous moistness, a respiratory rate, a heart rate, a postural change in heart rate, a blood pressure, a postural change in blood pressure, a venous pressure, a urine specific gravity, or any combination of these. The said body fluid may include saliva, sweat, tears, breath, urine, blood, or other transudates or exudates.

Typically, the first receiving system comprises an electronic-, chemical-, or electrochemical-based diagnostic or monitoring device. In some embodiments, the receiving system is calibrated according to a level of hydration having a numerical form, a quanlitative form, or a combination of these. The system may further comprise a second receiving system for receiving a patient's preference data wherein the processor calculates the quantity of the at least one beneficial agent that is to be released to the reservoir utilizing the received measured biometric data and the patient's preference data, wherein said patient preference data includes color, flavor, or amount of or inclusion or exclusion of a type of the at least one beneficial agent. The system may further comprise a third receiving system for receiving a patient's additional patient data wherein the processor calculates the quantity of the at least one beneficial agent that is to be released to the reservoir utilizing the received measured biometric data, the patient's preference data, if applicable, and the patient's additional patient data, wherein said additional patient data includes rate or amount of fluid loss, rate or amount of electrolyte loss, fluid electrolyte concentration, fluid osmolality, prior or expected future fluid consumption, prior or expected future electrolyte consumption, weight, weight gain or loss, body mass index, sex, age, race, height, behavioral data, diet, fitness level, physical exertion level, physical appearance, cognitive capability, environmental data such as temperature, humidity, altitude, the level of other disease markers, medication and medical procedure history, the presence, absence of severity of one or more particular medical conditions, and any combination of these. The processor may refine the calculation of the quantity of the at least one beneficial agent that is to be released to the reservoir by utilizing previously-recorded patient biometric data and/or trends in previously-recorded patient biometric data.

In any case, the receiving systems may receive manually input patient data. In such instances, the system may include a pressable button, pressable arrows, a pushable or pullable lever or a turnable wheel for manually inputting the patient data. In other embodiments, the receiving systems are configured to receive the biometric data from a diagnostic or monitoring device via electronic or wireless transmission. The processor may generate at least one electronic command which actuates the delivery system.

In some embodiments, the processor calculates the quantity utilizing known patient averages. For example, the known patient average may include total body water as a percentage of total body weight, a rate or amount of fluid loss, a rate or amount of electrolyte loss, and fluid electrolyte concentration or osmolality. In other embodiments, the processor calculates the quantity utilizing a patient's selection of a stock keeping unit (SKU).

It may be appreciated that the delivery system may include a door, lever, sieve or syringe-like device which is manipulated to release the beneficial agent. Further, the at least one beneficial agent contained in the container may be separated into discrete doses, and the delivery system may be configured to release one or more discrete doses. In other embodiments, the delivery system may be configured to release the quantity all at once or in stages over time.

In some embodiments, the reservoir is configured to orally administer the aqueous component and quantity of the at least one beneficial agent to the patient directly, through a spout structure mounted onto the reservoir or through a straw-like device.

Although the foregoing invention has been described in some detail by way of illustration and example, for purposes of clarity of understanding, it will be obvious that various alternatives, modifications and equivalents may be used and the above description should not be taken as limiting the scope of the invention which is defined by the appended claims.

All publications and patent applications cited in this specification are herein incorporated by reference as if each individual publication or patent application were specifically and individually indicated to be incorporated by reference.

What is claimed is:

1. A method for providing a hydration fluid to a patient, said method comprising:
determining a level of hydration of the patient; and
preparing a hydration fluid by combining an amount of a soluble hydration agent with an aqueous component, wherein the amount and/or type of hydration agent is selected based on the patient's determined level of hydration.

2. A method as in claim 1, wherein determining the level of hydration comprises measuring sodium concentration or osmolality of a bodily fluid and/or biomarkers for sodium concentration or osmolality of a bodily fluid.

3. A method as in claim 2, wherein the bodily fluid comprises saliva, oral fluids, sweat, tears, breath, urine, transudates, exudates, or blood.

4. A method as in claim 2, wherein determining the level of hydration further comprises utilizing at least one known patient average.

5. A method as in claim 4, wherein the at least one known patient average comprises total body water as a percentage of total body weight, fluid electrolyte concentration, a rate or amount of fluid loss, a rate or amount of electrolyte loss, and any combination of these.

6. A method as in claim 2, wherein determining the level of hydration further comprises utilizing additional patient data.

7. A method as in claim 6, wherein said additional patient data comprises rate or amount of fluid loss, rate or amount of electrolyte loss, fluid electrolyte concentration, fluid osmolality, prior or expected future fluid consumption, prior or expected future electrolyte consumption, weight, weight gain or less, body mass index, sex, age, race, height, behavioral data, diet, fitness level, physical exertion level, physical appearance, cognitive capability, environmental data such as temperature, humidity, and altitude, the level of other disease markers, medication and medical procedure history, the presence, absence or severity of one or more particular medical conditions, and any combination of these.

8. A method as in claim 2, wherein determining the level of hydration further comprises utilizing patient biometric data and/or previously-determined patient biometric data and/or trends in patient biometric data.

9. A method as in claim 1, wherein the amount and/or type of hydration agent is further selected based on a patient preference.

10. A method as in claim 1, wherein preparing the hydration agent comprises releasing the amount of soluble rehydration agent from a container which is integral with, or mounted or mountable on or in, a reservoir which holds the aqueous component.

11. A method as in claim 1, wherein the reservoir comprises a cup, bottle, bladder, tube, box, can, sack, thermos, canister, soft gu-like pouch, nursing bottle, covered children's cup, rehydration container, drinking-straw-type tube, or an on-demand drinking apparatus.

12. A method as in claim 1, wherein the reservoir is comprised of a hard material, a soft material, plastic, glass, aluminum, stainless steel, rubber or a combination thereof.

13. A method as in claim 1, wherein the reservoir includes a drinking spout through which the patient may drink.

14. A method as in claim 13, wherein the container is removably attached to, formed integrally with, or detached from the reservoir or drinking spout.

15. A method as in claim 1, wherein the at least one hydration agent is present in the container as one or multiple doses and wherein the one or multiple doses can be individually released into the aqueous component.

16. A method as in claim 1, wherein preparing the hydration fluid comprises mechanically releasing a calibrated amount of the hydration agent into the aqueous component held in a reservoir.

17. A method as in claim 16, wherein mechanically releasing a calibrated amount of agent is accomplished by advancing a pusher plate or bar, turning a dial, or depressing a blister mechanism.

18. A method as in claim 1, wherein preparing the hydration fluid comprises pouring or dropping a measured amount of the hydration agent into the aqueous component held in a reservoir.

19. A method as in claim 1, wherein the hydration agent comprises a powder or granulated solid, a dissolvable tablet, a plurality of encapsulated pellets, a liquid or gel.

20. A method as in claim 1, wherein the hydration agent is disposed in a pouch or osmotic device or is formulated in a controlled-release dosage form.

21. A method as in claim 1, wherein the hydration agent is selected from the group consisting of electrolytes, rehydration solutions, carbohydrates, nutrients, ergogenic supplements, medicaments, probiotics, and diagnostic agents.

22. A method as in claim 21, wherein the hydration agent comprises an electrolyte selected from the group consisting of sodium, potassium, calcium, magnesium, salts of chloride, bicarbonate, phosphate, and sulfate.

23. A method as in claim 21, wherein the hydration agent comprises a carbohydrate selected from the group consisting of sucrose, glucose, fructose, glucose polymers, and maltodextrins.

24. A method as in claim 21, wherein the hydration agent comprises a nutrient selected from the group consisting of glutamine, hydrolysates, vitamins, minerals, proteins, amino acids, polyamines, arginine, oligosaccharides, short chain fatty acids, enzymes, soluble fibers, fermentable fiber, phytochemicals, pyruvamide, pyruvyl-amino acids, amides of pyruvyl-amino acids, pyruvates, esters, salts, structured lipids, fats, d-chiroinositol, lactoferrin, marine oils, acidulents, ascorbic acid, and anti-oxidants.

25. A method as in claim 21, wherein the hydration agent comprises an ergogenic supplement selected from the group consisting of creatine, carbohydrates, caffeine, carnitine, bicarbonate, glycerol, antioxidant vitamins, colostrums, glutamine, amino acids, inosine, coenzyme QlO, herbs, beta-Hydroxy-beta-MethylButyrate, chromium, choline, GHB, vanadyl sulphate, and hydroxymethylbutyrate.

26. A method as in claim 21, wherein the hydration agent comprises a medicament selected from the group consisting of medications for diabetic conditions, chemotherapy agents, gastrointestinal drugs, antacids, antibiotics, probiotic medications, prokinetic medications, bioactive peptides, antihistamine drugs, anti-infective agents, antivirals, urinary tract anti-infectives, antineoplastic agents, autonomic drugs, adrenergic agents, skeletal muscle relaxants, blood formation drugs, coagulation drugs, cardiovascular drugs, central nervous system agents, diagnostic agents, electrolytic balance agents, caloric balance agents, water balance agents, enzymes, antitussive agents, expectorant agents, mucolytic agents, gold compounds, hormones, synthetic substitutes, smooth muscle relaxants, H.sub.2 blockers, Tagamet.RTM, and consistency-altering agents.

27. A method as in claim 21, wherein the hydration agent comprises a probiotic selected from the group consisting of Lactobacillus reuteri, Lactobacillus acidophilus, Lactobacillus animalis, Lactobacillus salivarius, or a live or dead microbial food supplement which affects the host's microbial balance in the gastrointestinal tract.

28. A method as in claim 1, wherein preparing the hydration fluid further comprises combining a flavoring agent in the aqueous component.

29. A drinking system comprising:
a reservoir containing an aqueous component; and
means integral with or attached or attachable to the reservoir combining a measured amount of soluble beneficial agent with the aqueous component.

30. A drinking system as in claim 29, wherein the combining means comprises a container integral with or mounted or mountable on or in the reservoir, said container holding at least one soluble beneficial agent.

31. A drinking system as in claim 29, further comprising a first receiving system for receiving a patient's measured biometric data, said receiving system calibrated as a function of at least one biometric data type; and a processor which calculates a quantity of the agent that is to be released to the reservoir utilizing the received measured biometric data.

32. A drinking system as in claim 31, wherein the combining means releases the quantity of agent calculated by the processor.

33. A drinking system as in claim 32, further comprising additional receiving systems for receiving additional patient data and/or patient preferences, wherein the processor calculates the quantity of the at least one beneficial agent that is to be released to the reservoir utilizing the biometric data together with the additional patient data and/or patient preferences.

34. A drinking system as in claim 33, wherein said receiving systems receive manually input patient biometric data, patient preference data, and additional patient data.

35. A drinking system as in claim 34, wherein said additional patient data comprises rate or amount of fluid loss, rate or amount of electrolyte loss, fluid electrolyte concentration, fluid osmolality, prior or expected future fluid consumption, prior or expected future electrolyte consumption, weight, weight gain or loss, body mass index, sex, age, race, height, behavioral data, diet, fitness level, physical exertion level, physical appearance, cognitive capability, environmental data such as temperature, humidity, and altitude, the level of other disease markers, medication and medical procedure history, the presence, absence or severity of one or more particular medical conditions, and any combination of these.

36. A drinking system as in claim 33, wherein the processor calculates the quantity utilizing known patient averages and/or previously-recorded patient biometric data or trends in previously-recorded patient biometric data.

37. A drinking system as in claim 36, wherein the known patient average comprises total body water as a percentage of total body weight, fluid electrolyte concentration, a rate or amount of fluid loss or a rate or amount of electrolyte loss.
